(19) United States
(12) Patent Application Publication
(10) Pub. No.: US 2011/0172503 A1
(43) Pub. Date: Jul. 14, 2011

(54) Physiological Data Collection System

(75) Inventors: Michael B. Knepper, Friedens, PA (US); Noam Hadas, Tel-Aviv (IL)

(73) Assignee: SUNRISE MEDICAL HHG, INC., Longmont, CO (US)

(21) Appl. No.: 12/668,765

(22) PCT Filed: Jul. 16, 2008
(86) PCT No.: PCT/US2008/070153

§ 371 Date: Jan. 31, 2011

Related U.S. Application Data
(60) Provisional application No. 60/959,745, filed on Jul. 16, 2007.
(60) Provisional application No. 60/959,746, filed on Jul. 16, 2007.
(60) Provisional application No. 60/959,747, filed on Jul. 16, 2007.
(60) Provisional application No. 60/959,748, filed on Jul. 16, 2007.

(51) Int. Cl. A61B 5/00

(52) U.S. Cl. 600/301

(57) ABSTRACT

A physiological data collection system that includes a recorder box having a memory device. The recorder box is in communication with a plurality of external sensors and a plurality of internal sensors. The physiological data collection system further includes a speaker and a controller, each in communication with the recorder box. The controller is provided for controlling the operation of the recorder box. The physiological data collection system further includes a set of ancillary functions that support and improve data integrity, usability, cost effectiveness and reliability of the system.

### CROSS-REFERENCE TO RELATED APPLICATIONS

This application claims the benefit of U.S. Provisional Application No. 60/959,745, filed Jul. 16, 2007; U.S. Provisional Application No. 60/959,746, filed Jul. 16, 2007; U.S. Provisional Application No. 60/959,747, filed Jul. 16, 2007; and U.S. Provisional Application No. 60/959,748, filed Jul. 16, 2007, the disclosures of which are incorporated herein by reference.

### TECHNICAL FIELD

The invention relates generally to medical diagnostic systems. More specifically, the invention is directed to a physiological data collection system.

### BACKGROUND OF THE INVENTION

Physiological data collection systems are used to collect and process data concerning the physiological parameters of patients in many types of diagnostic procedures. These systems use electronic recorders to collect, store and produce information concerning patterns such as respiration, motion, electrophysiological parameters and similar data. Many types of data can be recorded by these systems. For example, information regarding body movement, body physiology, and external events can be gathered.

### BRIEF SUMMARY OF THE INVENTION

The invention relates to a physiological data collection system. In an embodiment of the invention, the physiological data collection system includes memory devices, a plurality of internal and external sensors, and a controller for controlling the operation of a recorder box. The operation of the recorder box is further augmented by features and devices which improve performance, patient compliance, and data reliability and coherence; along with increased utility.

### BRIEF DESCRIPTION OF THE DRAWINGS

FIG. 1 is a schematic view of a physiological data collection system according to the invention;

FIG. 2 is a schematic view of the system of FIG. 1 positioned on a patient;

FIG. 3 is a front elevational view of a recorder box according to the invention;

FIG. 4 is a rear elevational view of the recorder box of FIG. 3;

FIG. 5 is a side elevational view of the recorder box of FIG. 3;

FIG. 6 is an exploded view of a memory device and the recorder box of FIG. 3;

FIG. 7 is an enlarged, perspective view showing a memory interface of the recorder box of FIG. 3;

FIG. 8 is a schematic view of an oximetry probe according to the invention;

FIG. 9 is a schematic view of a communication link for a physiological data collection system according to the invention; and

FIG. 10 is a schematic view of data output of a physiological data collection system according to the invention.

### DETAILED DESCRIPTION OF THE INVENTION

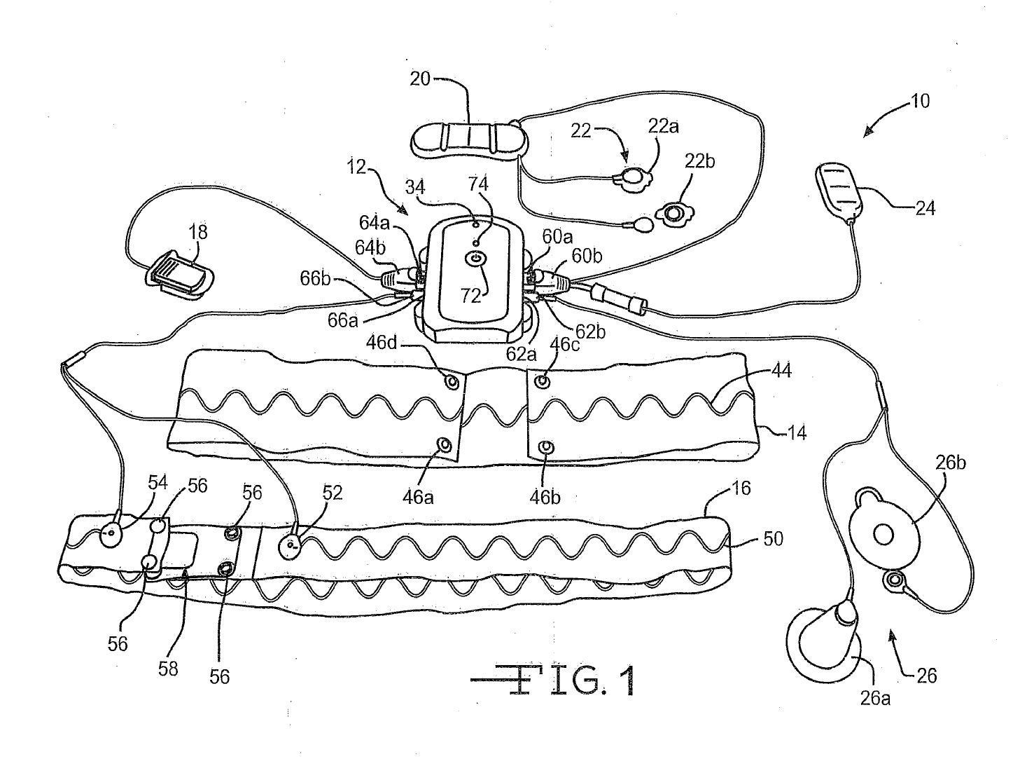

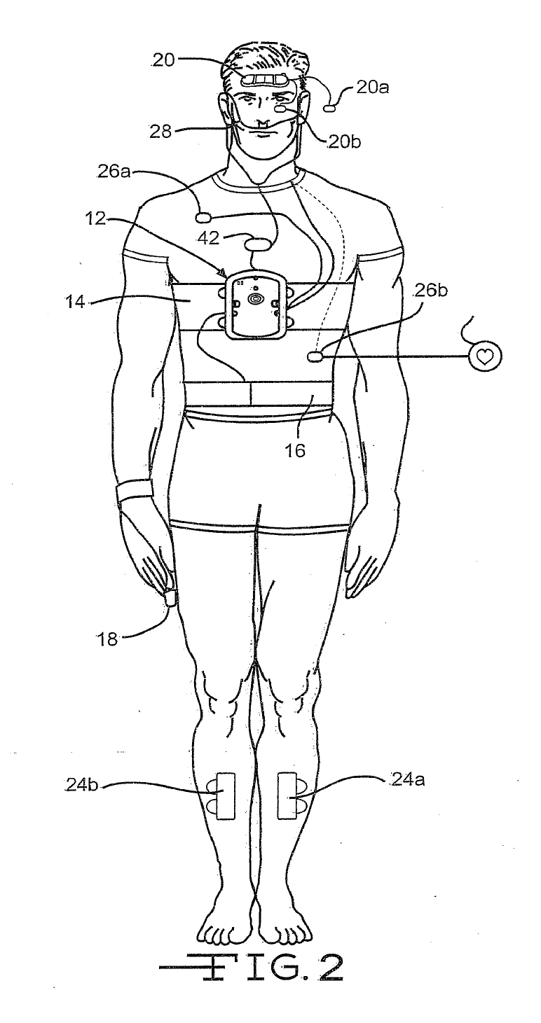

Referring now to the drawings, a physiological data collection system according to the invention is indicated generally by the reference number 10. Referring to FIGS. 1 and 2, the physiological data collection system 10 includes a recorder box 12 for recording physiological signal information. In an embodiment, the recorder box 12 is in communication with a plurality of external sensors through a plurality of external channels. The external sensors may include, for example, a chest effort belt 14, an abdominal effort belt 16, an oximetry probe 18, and a plurality of other external sensors adapted to monitor or measure the functional state or activity of various bodily and internal organs. The plurality of sensors for measuring internal organ function includes an electroencephalogram (EEG) sensors 20 for monitoring electrical brain activity, electrooculogram (BOG) sensors 22 for monitoring eye movement (two are shown), electromyogram (EMG) sensors 24 for monitoring muscular activity, and electrocardiogram (ECG) sensors 26 for monitoring cardiac activity. The physiological data collection system 10 may further include a nasal cannula 28 that is in communication with the recorder box 12. The nasal cannula 28 may be in communication with an internal pressure sensor 30 for monitoring respiration through pressure changes in the nasal cavity. The external sensors are shown to be in wired communication with the recorder box 12. Alternatively, some or all of the external sensors may be in wireless communication with the recorder box 12.

FIG. 2 shows the physiological data collection system 10 positioned on a patient. The EOG sensor 20 can be first and second EOG sensors 20a and 20b. For example, the first EOG sensor 20a may be positioned below the patient's left eye and the second EOG sensor 20b may be positioned above the patient's right eye. The EMG sensor 24 may be first and second EMG sensors 24a and 24b, shown in FIG. 2 on the patient's legs. Alternatively, the EMG sensor 24 may be a facially applied sensor such as, for example, a single chin EMG sensor. The chin EMG sensor monitors signals associated with certain facial muscular movements. The ECG sensor 26 maybe. first and second ECG sensors 26a and 26b placed on the patient's chest.

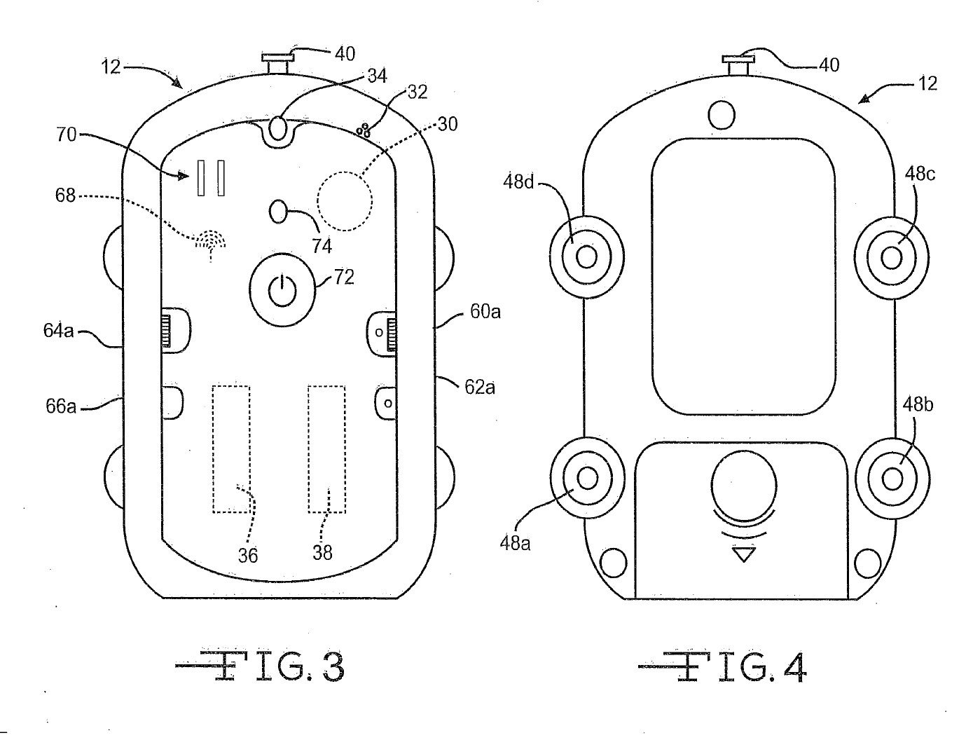

Referring to FIG. 3, the physiological data collection system 12 may also include a plurality of internal sensors. In an embodiment, the recorder box 1.2 may include the pressure sensor 30 that is in communication with the nasal cannula 28 (shown in FIG. 2). The internal sensors further include a microphone 32, a photo detector 34 to measure ambient light levels, a spatial position sensor 36, and a body movement sensor 38. The function of the spatial position sensor 36 may be integrated into the body movement sensor 38.

The pressure sensor 30 measures breathing pressure and/or breathing flow rate transmitted by the nasal cannula 28 through a pressure connection port 40. In an embodiment, the pressure connection port 40 is in fluid communication with the pressure sensor 30. The pressure sensor 30 may also monitor pressure output of a continuous positive airway pressure (CPAP) device. The pressure connection port 40 may be configured as a female port or luer, for example a 0.107 inch luer connector, for fluid coupling of the cannula 28 to the recorder box 12. The cannula 28 may include a mating male luer (not shown) and an in-line, disposable hydrophobic filter 42, as shown in FIG. 2.

The microphone 32 is defined herein as a voice recording module that includes a voice recording circuit, a supporting software or algorithm that includes a mode selection portion, and a microphone element. The microphone element may be provided as, for example, an electret microphone, though any other device suitable to convert acoustical waves into an electrical signal may be used. The microphone 32 may be operated in two operational modes, a first recording mode and a second recording mode. The first recording mode is a patient-activated mode that allows the patient to record messages related to spurious events such as, for example, bathroom use. The second recording mode is a continuous monitoring mode for collecting ambient noise during the physiological study session including, for example, patient snoring.

When the microphone 32 is operated in the first recording mode, the patient may initiate the recording of a voice message during an event for a predetermined period of time such as five seconds, or until the patient stops talking for a predetermined period such as two seconds. The message is recorded on the memory media together with a real-time stamp and can be correlated in time with the physiological data traces. This correlated information provides an indication and supporting information to an interpreter of the study results that the physiological information recorded in the temporal vicinity of the recorded event message had an anomaly or special characteristic based on the event. The microphone and related supporting software may be fitted in an ECG Holter recorder, allowing the patient to record messages such as “I just had to run after a bus.” The message allows the interpreter to explain why a sudden increase in heart rate is apparent a few seconds after the message. The microphone 32 of the physiological data recorder box 12 can also be used, for example, during use of the recorder in a sleep study to alert the technician reviewing the study that the patient needed to go to the bathroom, or was awakened by a dog barking in the street.

In the second recording mode, the microphone 32, may operate in a continuous recording mode. The continuous recording mode may record ambient noise, interrupted by the patient-activated mode.

The microphone 32 of the physiological data recorder box 12 may also be used at the beginning of the study for identification purposes. Coordinated identification of the patient to the recorded data helps ensure that a recording extracted from the memory of a specific recorder or memory device is the physiological data of a specific patient. This identification capability minimizes concerns of recorders being mixed-up at the dispensing or the downloading stations. The microphone 32 may therefore be used to have the patient record his name and I.D. number, in his own voice onto the physiological data file and linked to the physiological data, allowing assured identification of each file.

The photo detector 34 senses ambient light levels during the physiological study. The photo detector 34 may be physically integrated into the recorder box 12 such that the sensed light level is recorded for later playback and data manipulation. In an embodiment of the invention, the photo detector 34 may be a singular sensor or a plurality of various sensors that sense a variety of associated ambient conditions, or other information, which may not be physiological in nature. These sensors may be integrated in the physiological data recording system 10. Such ambient sensors may include an ambient light or light spectral distribution sensor, a relative humidity sensor, a temperature sensor, a noise level sensor, an air pollution level sensor, a barometric pressure sensor, a radiation sensor (either in the visible range, infrared or UV range, microwaves, or any other type of radiation), acceleration and inclination, wind speed or any other sensor that responds to parameters outside of the patient. The signals received from these sensors, such as the photo detector 34, may also be correlated in time with the physiological sensor data traces to provide an indication, to an interpreter of the study results, that the trace patterns may have been affected by these external conditions to which the patient was exposed during the study.

The body position sensor 36 may be integrated inside the recorder box 12 to detect a patient's body position in all three spatial axes. Alternatively, the body position sensor 36 may be a software function that derives body position in three spatial axes from two channel inputs of the body movement sensor 38. The body movement sensor 38 utilizes two channels of the gravity-referenced, accelerometer measurements to derive the body position in all three axes. In an embodiment, the body movement sensor 38 is an internally mounted DC response accelerometer. The two channel accelerometer is oriented and mounted in the recorder box 12 such that a signal output in one channel is proportional to the vector of gravity superimposed on the front to back (Sagital) axis, and on the left to right (Frontal) axis in the other channel. The accelerometer orientation may be associated with an orientation of the recorder relative to the patient as provided by the user instructions. The general orientation of the body may be calculated from trigonometric relationships using these two values. The software analyzing these channels may derive full three axis orientation data by utilizing an algorithm to assess and rule out body positions which are physically impossible or improbable to achieve such as, for example, bending backwards when standing, or head and torso raising from the bed when lying in a prone position.

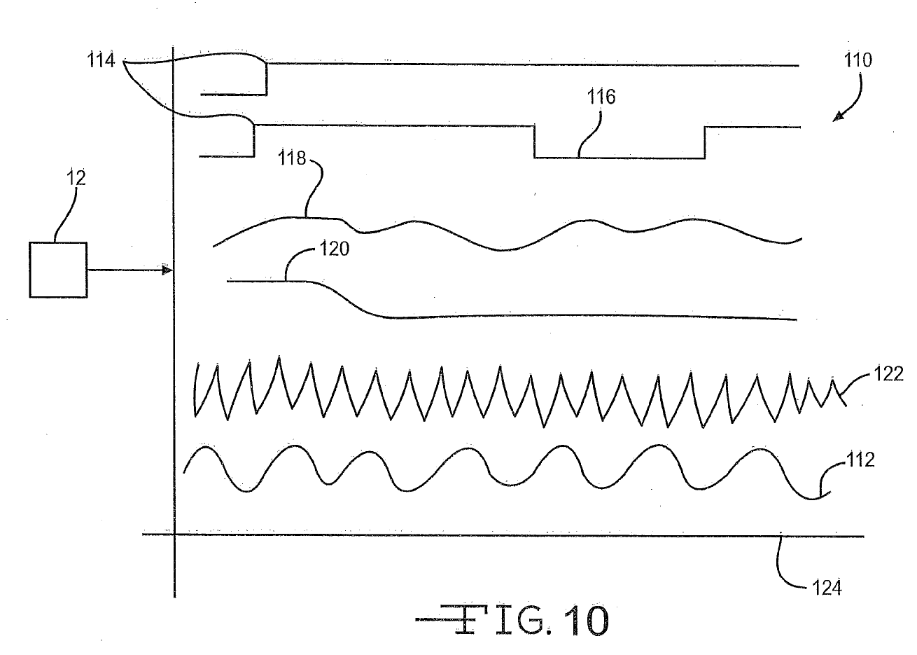

In an embodiment of the physiological data collection system 10, such as that used in sleep studies, the recorder box 12 may be applied on the patient's body, as shown in FIGS. 2 and 9. This mounting configuration eliminates the need for cables leading from various sensors attached to the patient to the recorder box, which is situated on the night stand or hanging on the wall. In many of these applications, respiration, as monitored or measured through measuring the expansion of the chest or abdomen cavity, is a specified parameter to be recorded. The same sensor, in the shape of a band strapped around the patients' body, may be used to monitor chest or abdomen expansion with respiration and simultaneously provide the mechanical attachment for securing the recorder box at the desired location on the body. The chest effort belt 14 as shown in FIG. 1 is made of a resilient material sufficient to adjust to the expansion and contraction of the chest cavity during breathing. The belt 14 is also sufficiently stiff to support the weight and orientation of the box when the patient moves. In one embodiment of such a sensor, the belt 14 includes a conductive element 44 such as a metallic, insulated or non-insulated wire that may be interwoven or attached to the band in another way that will not interfere with its elastic nature. The area enclosed by the closed loop foamed by the conductive element 44 moves with the belt 14 and therefore changes inductance as the patient's chest expands and contracts. The changing inductance provides an electrical measurement of the expansion and contraction of the chest during the study to determine the breathing effort associated with the patient.

The chest effort belt 14 includes a plurality of chest belt attachment points 46a, 46b, 46c, and 46d. Though shown as four attachment points, however, there may be more or less in number. At least two of the attachment points such as points 46a and 46b may also serve as electrical contacts that are in electrical communication with the conductive element 44. The attachment points 46a and 46b provide both electrical connectivity and mechanical attachment between the chest belt 14 and the recorder box 12. Further, the belt 14 and attachment points 46a, 46b, 46c, and 46d secure the recorder box 12 to the patient sufficiently so that the internal sensors may provide accurate data, for example, data collected by the body position sensor 36 pertaining to patient movement and sleeping position. The attachment points 46a-46d are illustrated as fabric snap-type fastener connections in which the attachment points 46a and 46b are also electrically conductive.

As shown in FIG. 4, the recorder box 12 has corresponding mating recorder connection points 48a, 48b, 48c, and 48d. The recorder attachment points 48a-d engage and connect to the belt attachment points 46a-d to provide both securement and electrical communication therebetween. For example, mating points 48a and 48b may be electrically connected to the internal circuitry of the recorder box 12 for communication therebetween. The corresponding electrical belt points 46a and 46b electrically couple the belt 14 to the recorder box 12 and the internal circuitry. The remaining points 46c, 46d, and 48c, 48d engage each other, respectively, to support and engage the recorder box 12 to the patient's chest. The belt and recorder attachment points 46a-d and 48a-d are illustrated as fabric snap-type fasteners, though any suitable load-bearing and electrical connection may be used.

The physiological data collection system 10 may also include an additional signal self-test function intended to increase its applicability, usefulness, and signal reliability. Embedded in the recorder software there may be a routine or algorithm that can perform signal quality checks on the signals from all externally applied sensors and accessories. These checks may be performed using one or more of three possible strategies. The system 10 may either perform periodic checks, for example, every fifteen minutes, and stop the recording to analyze a short data section already recorded in the system memory. This analysis provides a decision, if any is needed, as to whether the recorded signals show signs of a defective or misplaced sensor. The algorithm may also analyze signal quality by comparing values derived from different channels. The different channels provide an alternative perspective of the same physiological parameter by way of different physiological routes—such as heart rate that is derived from an optical plethysmographic signal and ECG signals.

Alternatively, the software may stop recording, but continue to collect and analyze the signals to arrive at the same decision. Thus, an error will be indicated only if it is present at the time of the test. A third possibility is that the software performs all signal quality tests at the same time as recording them in memory. This strategy provides real time indication of errors for an increase in computational resources.

The abdominal effort belt 16 as shown in FIG. 1 is made of a resilient material sufficient to adjust to the expansion and contraction of the patient's abdomen during breathing. In an embodiment, the belt 16 includes an abdominal conductive element 50 such as a sinusoidally applied wire that may be interwoven with the belt 16 or applied to the surface thereof. The operation of the abdominal effort belt 16 is similar to the chest effort belt 14. The abdominal conductive element 50 terminates in first and second contacts 52 and 54. The abdominal effort belt 16 further includes fabric connectors such as fabric snaps 56 and an adjustment buckle 58 that may be a hook-and-loop connection. The adjustment buckle 58 allows one size of abdominal effort belt 16 to accommodate a range of patient sizes.

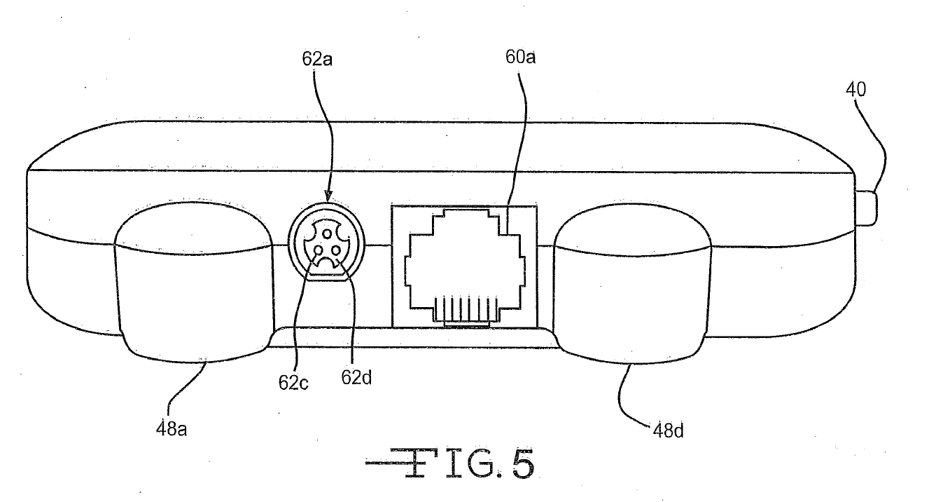

Referring now to FIGS. 1, 3, and 5, a plurality of external connection points are illustrated that couple the various external sensors to the recorder box 12. Though illustrated and described as specific connector types, any connector that functions to communicate between the external sensor or sensors and the recorder box 12 may be used. In an embodiment, a first connector 60a and a second connector 62a are positioned on one side of the recorder box 12. The opposite side of the recorder box 12 includes a third connector 64a and a fourth connector 66a.

In an embodiment, the first connector 60a and the third connector 64a are female RJ45-type, eight pin/eight coupler connectors commonly used in telephony and computer communications and also commonly associated with Category-5 type twisted-pair wiring. The connector 60a connects the EEG sensor 20, the EOG sensors 22a and 22b, and the chin EMG sensor 24 to the recorder box 12 by way of a mating male RJ45-type connector 60b, as shown in FIG. 1. In an embodiment, the second connector 62a and the fourth connector 66a are configured as three-pin male safety connectors. The connector 62a includes three male pins 62c recessed in a female receptacle 62d. The second connector 62a connects the ECG sensors 26a and 26b to the sleep recorder by way of a mating three pin connector 62b, as shown in FIG. 1. The third and fourth connectors 64a and 66a couple the oximetry probe 18 and the abdomen effort belt 16, respectively, to the recorder box 12 by way of mating connectors 64b and 66b.

The single connector for multiple sensors functions as an easy-to-use, “poka yoke” device to ensure proper connection. The sensors may be grouped by various sensor characteristics such as similar functions, similar data post processing requirements, or similar sensor types. For example, the EEG sensor 20, EOG sensors 22a and 22b, and the chin EMG sensor 24 may be grouped together as facially applied sensors. The sensors, whether singular or grouped, are provided with corresponding, mating male or female connectors to couple to the recorder box 12. The external sensor connections may also be color coded to the external connection points of the recorder box 12 to further simplify proper identification and patient connection.

In an embodiment, as shown in FIG. 3, a wireless transmitter/receiver (WTR) unit 68 provides a plurality of communication channels to allow multiple sensors to operate wirelessly. The WTR unit 68 may provide eight separate communication channels, though more or less in number may be provided. For example, the EEG/EOG/facial EMG group sensors 20, 22a, 22b, and 24 may communicate with the recorder box 12 through the WTR unit 68. Alternatively, the EMG sensors 24a and 24b applied to the patient's legs may communicate wirelessly to facilitate walking.

Still referring to FIG. 3, there is provided within the recorder box 12 of the physiological data collection system 10 a speaker 70 that includes three output modes. The speaker 70 is defined herein as a voice messaging module that includes a voice reproduction circuit, a supporting software or algorithm, an audio amplifier, and a speaker element. A first speaker output mode may be a patient-introductory and setup instruction mode that provides verbal directions for various functions, set-ups, and operational characteristics of the physiological data collection system 10. For example, upon start up, audible instructions may be provided to the patient for applying the various sensors specified in the specific study protocol. The first speaker output mode can be used, for example, to guide an unskilled and unattended patient during setup for the study in the patient's home. The speaker 70 provides audio messages that may also be used to provide information concerning assembly, installation, and/or use of the physiological data collection system 10 and its related components. These instructions may be programmed to follow a pre-programmed study setup flow chart, which is automatically uploaded from a personal computer according to the indicated channels selected and study parameters. For example, the system 10 issues voice prompts to lead the patient through each step of the pre-recording set-up process. The speaker 70 uses the output from the self-check protocols to verify that the instructions for activating and applying the sensors were followed properly. The speaker 70 may further provide a message alerting the patient to readjust or correct his or her actions and checking again until the step or steps are accomplished successfully. This corresponds to the actions a trained technician would take in setting up the study.

A second speaker output mode may be one, or any combination, of a verbal alert, a tonal alert, and a vibratory alert. The second output mode is provided for signaling a condition, either an error condition or a use-ready condition, associated with the recorder box 12 or the various sensors. This second output mode may operate in conjunction with a sensor verification mode. As the patient initiates the physiological data collection system 10 and applies the sensors as required, the recorder box 12 performs an operational check of each sensor. If the sensors are not verified to be properly applied or in working order, an error condition is signaled. The recorder box 12 may be programmed to require sensor adjustment or replacement, or the recorder box 12 may continue on and bypass the malfunctioning sensor.

When operating in the second output mode, the system 12 responds to inputs from the sensor inspections conducted during the study. The system 12 may be programmed to wake the patient, stop recording, or continue recording if a sensor anomaly is detected. In the event a wake-mode is selected, the voice alert feature may output a wake-up alert, either verbal, tonal, vibratory, or any combination thereof to alert the patient that sensor attention is required. If a stop recording option is selected, the system will cease recording either the affected channel or the entire study depending on the programmed response. The system may also be programmed to ignore the error message and continue recording all sensor channels.

The system 10 may be programmed in an error correction instruction mode to issue a verbal warning to the patient that there is a problem with one or more of the sensors. The system 12 may further identify the problem and suggest a resolution. The system 12 may then check the sensor signal to confirm that the problem has been resolved and that the study can continue, or provide further instructions on how to correct the problem or any other measures that must be taken. In an embodiment of the physiological data collection system 10 configured as a sleep disorder recording system, the system may be programmed to awaken the patient when needed.

A third speaker output mode, or a special test condition instruction mode, of the voice alert feature allows the physician ordering the physiological recording to gather data in some specific situations of special interest to him. In this third mode the physician may program the system to instruct the patient to perform certain tasks at predetermined times in the study, or if certain conditions measured from the various sensors are met. As an example, in an embodiment as a sleep recording system, the voice messaging function may be used to ask the patient to move, for example, from a prone position to a supine position to allow for data collection in various positions.

As shown in FIG. 3, the recorder box 12 includes a push button 72 and an indicator light 74. The push button 72 initiates a plurality of functions including a system power on and power off function, an event marker and associated time stamp function, and a recording function. The recording function is coordinated with the event marker and time stamp function to help segregate sensor data that is potentially affected by the event. The push button 72 is hardwired and software programmed to provide the various functions. Holding the push button 72 for a period of time after the recorder box 12 is powered accesses the event marker subroutine of the data collection algorithm. The indicator light 74 provides status indication of power, sensor status, and recording operation. The indicator light 74 may further provide assistance to awaken and alert a patient that some action is required. The indicator light 74 may be any type of light such as, for example, a light emitting diode. The light 74 may further provide a plurality of colors or flashing sequences associated with different alerts or status indications.

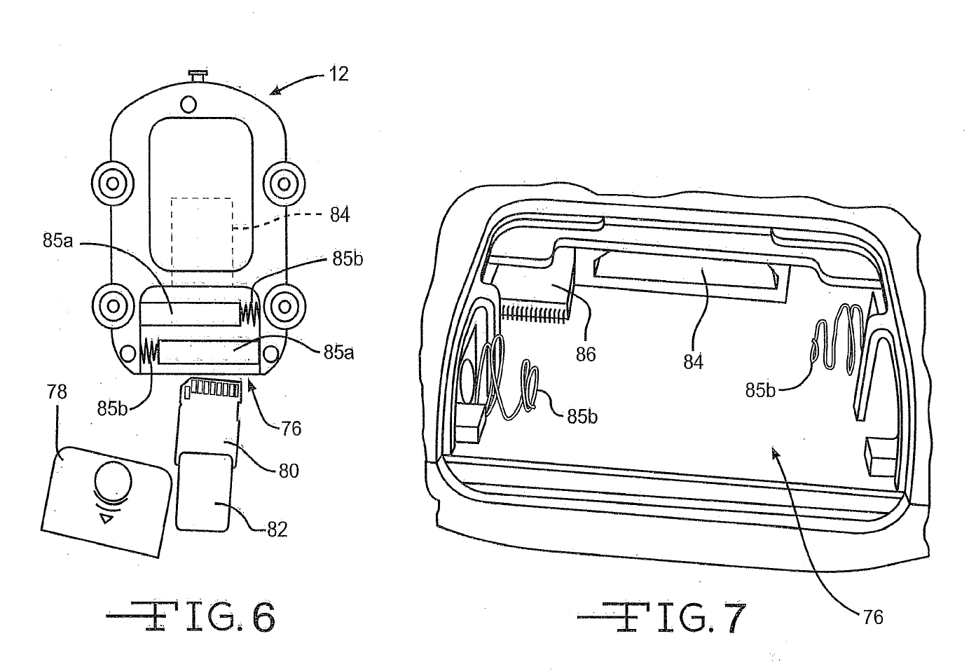

Referring now to FIGS. 6 and 7, the recorder box 12 includes a compartment 76, a compartment cover 78, and a memory device such as a smartcard 80. However, any memory device may be used such as, for example, a flash drive, a multimedia memory card, or a removable chip. The smartcard 80 may include a tag 82 attached thereto for written identification information and card removal purposes. The compartment 76 houses a card slot 84 that receives the smartcard 80 for communication with a controller such as a microprocessor 86. The compartment 76 can contain batteries 85a positioned between battery terminals 85b for providing power to the recorder box 12. The card slot 84 is further situated within the compartment 76 to provide a tamper evident function. The card slot 84 is located so that the batteries must first be removed in order to extract the smartcard. 80. Removal of batteries to access the smartcard 80, engaged in the slot 84, causes a disruption in the recording of data on the smartcard 80 by the controller 86. This tamper evident feature impedes removal of or prevents replacement of the smartcard 80 without health care provider or data interpreter knowledge.

Information collected by the various sensors selected for the sleep study is gathered by the controller 86 and recorded onto the smartcard 80. The smartcard 80 also contains prerecorded information such as, for example, patient identification information, sensor channel activation selections, clock setup information, and sound files associated with verbal prompts and alerts. These sound files may be generic or customized for the specific patient needs.

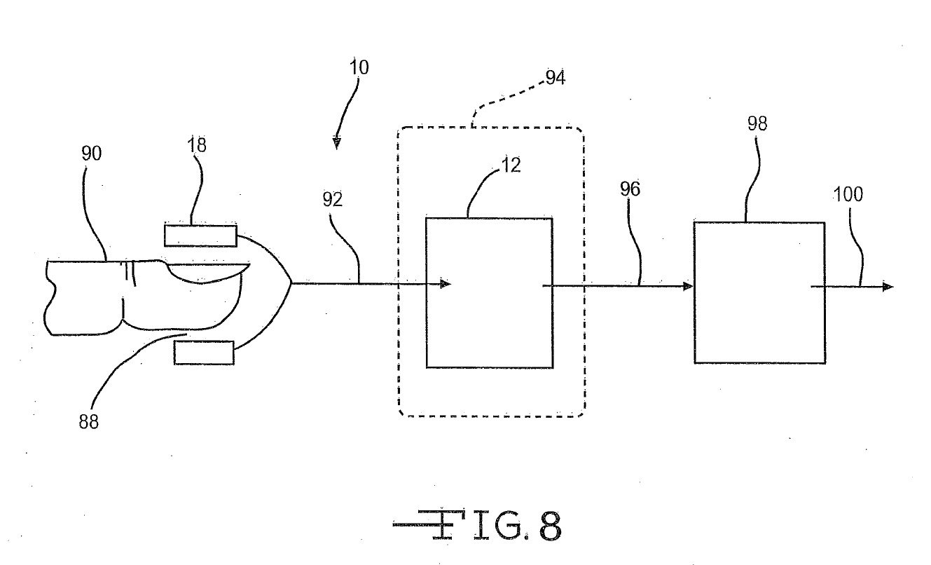

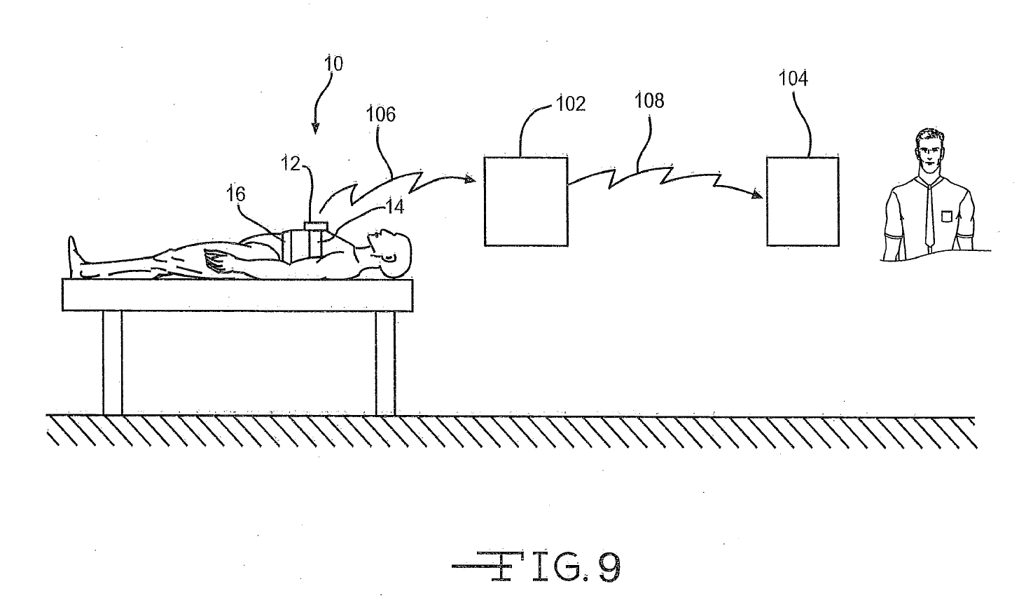

Referring to FIG. 8, the oximetry probe 18 includes an opening 88 for insertion of a patient's body part such as a finger 90, or it may be applied in a body part and use reflective radiation to provide similar information. In an embodiment, the probe 18 has a photo-sensor for converting optical signals into raw data from which various physiological values can be generated. The raw data of the optical signals may be sensor-generated signal data that is unprocessed or un-manipulated by post processing activities. For example, data concerning the percent saturation of oxygen in the blood of a patient, along with pulse rate, can be generated. As represented by arrow 92, the probe 18 transmits raw data without further processing to the recorder box 12. In an embodiment, the recorder box 12 can store raw data on the smartcard 80. The boundary of the recorder box 12 is shown by broken line 94.

As represented by arrow 96 in FIG. 8 the physiological data collection system 10 transmits the raw data to a selectable data processor such as a personal computer 98. The data transmission 96 may occur after the study is complete, if so desired. The raw data is processed by the computer 98 to calculate final physiological data such as, for example, saturation and pulse rate. The information stored in the data collection system may be the raw optical signal from the oximetry probe 18, rather than converted oximetry and pulse rate values calculated from the raw data, which is an industry common practice. The common data conversion practice typically utilizes special hardware and/or software modules in the recorder. This separation of the signal recording phase from the signal analysis phase provides advantages including lower part cost, lower cost of the circuit, and lower power consumption in the recorder box 12. Further more, improved processing capabilities in the computer 98, allow analytical algorithms to change in order to determine, for example, blood parameters without changing the hardware. Thus, access to the original signals is available when new processing techniques are developed that provide more accurate analyses. This separation of the data acquisition and analysis phases is applicable where no display or user interaction is required based on the derived physiological parameters. As represented by arrow 100 in FIG. 8, the computer 98 transmits processed and/or analyzed, data to another device. For example, this data can be transmitted to a storage device or a display device.

The sensor data processing such as, for example, pulse oximetry data processing can be separated into two phases: (1) collection and storage of information without manipulation and (2) analyzing the information at a later time. Accordingly, the analyzed information is not reviewed in real time. Instead, the raw information is reviewed by the computer 98 that may be, for example, a remote, off-line computer, which results in the above-described advantages.

It should be understood that the invention is not limited to sleep applications. For example, the invention can be used in Holter devices that monitor ECG, or measure pulse transit time, which store the ECG and optical pulse wave signal without any filtering and perform all calculations in post processing. Another example is use with peripheral arterial tone (PAT) signals.

Referring to FIG. 9, the recorder box 12 is in communication with the sensor or sensors such as, for example, the chest effort belt 14 and the abdominal effort belt 16. The sensors are attached to the patient undergoing the study, conducted either in the patient's home or a clinic setting. The physiological data collection system 10 further includes an automatic data analyzer and alarm transmitter 102 and an alarm receiver 104. In an embodiment, the alarm receiver 104 has a display device for visual alerts. In another embodiment, the alarm receiver 104 has a sounding device for audio alerts. In another embodiment, the alarm receiver 104 has both display and sounding devices. The alarm receiver 104 can be located away from the patient near an attendant to minimize sleep interference. For example, the alarm receiver 104 can be located in a nursing station near an attendant such as a nurse.

As represented by arrow 106 in FIG. 9, the recorder box 12 can transmit a signal to the data analyzer and alarm transmitter 102. As represented by arrow 108 in FIG. 9, the data analyzer and alarm transmitter 102 can transmit a signal to the alarm receiver 104.

In an embodiment, real time analysis of the input signals from one or more sensors placed on the patient will be conducted electronically on a regular interval or continuously in the recorder box 12. When sensor signal quality deteriorates, a signal can be transmitted to the alarm receiver 104 through the data analyzer and alarm transmitter 102. Upon receipt of the signal, the alarm receiver 104 can provide a visual and/or an audio alert to the attendant concerning the status of the sensor.

The advantages of the recorder box 12 with data analyzer and alarm transmitter 102 and the alarm receiver 104 include efficiency because the attendant has the ability to monitor more than one patient at the same time, lower cost due to automation of the determination of signal failure, and minimization of patient interference as a result of the positioning of the alarm receiver 104 in a location remote from the patient or patients.

Referring to FIG. 10, the recorder box 12 can record information as shown in a graph 110. For example, the information can include physiological signal traces 112, sensor active point 114 in which amplitude reflects quality of signal, sensor interruption 116, ambient noise 118, ambient light 120, and a recorded message indicator 122. The information can also include, for example, ambient temperature, air pressure, relative humidity, vibrations, smells and the presence of other people. In an embodiment, axis 124 indicates time.

While the invention has been described with reference to particular embodiments, it should be understood that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiments, but that the invention shall include all embodiments falling within the scope of the claims.

What is claimed is:

1. A physiological data collection system comprising:
a recorder box having a memory device;
a plurality of external sensors in communication with the recorder box;
a plurality of internal sensors in communication with the recorder box;
a speaker in communication with the recorder box; and
a controller in communication with the recorder box for controlling the operation of the recorder box.

2. The physiological data collection system of claim 1 wherein the recorder box includes a wireless transmitter/receiver channel.

3. The physiological data collection system of claim 1 wherein the memory device is a smartcard.

4. The physiological data collection system of claim 3 wherein the recorder box includes a slot in communication with the smartcard, the slot being positioned in the recorder box to impede the removal of the smartcard.

5. The physiological data collection system of claim 1 wherein the plurality of external sensors is selected from the group consisting of a chest effort belt, an abdominal effort belt, an electroencephalogram (EEG) sensor, an electrooculogram (EOG) sensor, an electromyogram (EMG) sensor, an electrocardiogram (ECG) sensor, an oximetry probe, and a nasal cannula.

6. The physiological data collection system of claim 5 wherein the chest effort belt includes a fastener for attaching the chest effort belt to the recorder box, the fastener being in electrical communication with the recorder box.

7. The physiological data collection system of claim 5 wherein the electroencephalogram (EEG) sensor, the electrooculogram (EOG) sensor, and the electromyogram (EMG) sensor are facially applied sensors having a common connector engaging a single external communication port for communication with the recorder box.

8. The physiological data collection system of claim 7 wherein the single external communication port is a female telephony port and the common connector is a male telephony connector.

9. The physiological data collection system of claim 1 wherein the plurality of internal sensors is selected from the group consisting of a microphone, a body movement sensor, a body position sensor, a pressure-flow sensor, and a multitude of ambient condition sensors.

10. The physiological data collection system of claim 9 wherein the microphone includes a patient-activated recording mode for recording a patient message and synchronizing a time stamp with the patient message, and a continuous monitoring mode for recording ambient sound.

11. The physiological data collection system of claim 9 wherein the body movement sensor is a DC response accelerometer, the body movement sensor having a body position function capable of determining body position in three axes.

12. The physiological data collection system of claim 1 wherein the speaker is contained within the recorder box, the speaker having one or more of a patient introduction and setup instruction mode, an error correction instruction mode, and a special test condition instruction mode, the speaker further providing an output of one of a verbal alert mode, a tonal alarm mode, and a vibratory mode.

13. The physiological data collection system of claim 1 wherein the controller includes an automatic signal quality evaluation process for checking sensor signal quality and processes the detection and recordation of sensor status, the controller further determining an output function including one of an alert signal, a wake up signal, and a continue recording instruction.

14. The physiological data collection system of claim 1 wherein the controller includes an auditory instruction guide that directs patients through an error detection process, an error correction process, and a re-initiation process, the controller further providing an output to the speaker that includes an alarm signal if a sensor signal output quality drops below a predetermined level.

15. The physiological data collection system of claim 1 wherein the controller records physiological sensor-generated signal data onto the memory device.
